(19) United States
(12) Patent Application Publication
(10) Pub. No.: US 2009/0104252 A1
(43) Pub. Date: Apr. 23, 2009

(54) WOUND DRESSING FOR IMPROVING SCARRING

(75) Inventors: Murad ALAM, Chicago, IL (US); John Y.S. KIM, Chicago, IL (US); Lucile E. White, Houston, TX (US)

Correspondence Address: DRINKER BIDDLE & REATH LLP;ATTN: PATENT DOCKET DEPT., 191 N. WACKER DRIVE, SUITE 3700, CHICAGO, IL, 60606, US

(73) Assignee: SCAR CONSULTANTS, LLC, Chicago, IL (US)

(21) Appl. No.: 11/875,654

(22) Filed: Oct. 19, 2007

(51) Int. Cl. A61K 33/38; A61F 13/02; A61P 17/02

(52) U.S. Cl. 424/446; 424/618

(57) ABSTRACT

An occlusive wound dressing for application to wound sites to accelerate the healing process and improve the appearance of the final mature scar including a silicone gel sheet and antimicrobial silver disposed in the silicone gel sheet where the silver may be present in the form of particles or wires and the particles or wires may be disposed in the silicone gel sheet in a grid pattern or as a lattice, and an adhesive may be present on at least one face of the silicone gel sheet. The adhesive may be present in the form of spaced lines with adhesive-free areas between the lines containing the antimicrobial silver. It is preferred that an antibruising agent is also included in the silicone gel.

### FIELD OF THE INVENTION

The present invention relates generally to occlusive wound dressings intended to modify the wound healing process at a skin wound site. More particularly, the present invention relates to silicone gel sheets containing agents with antibacterial and bruise-resolving properties that accelerate the healing process and improve the appearance of the final mature scar at a skin wound site.

### BACKGROUND OF THE INVENTION

Scar tissue is composed mainly of the fibrous protein collagen formed during the biologic process of wound repair of the skin. Scarring is thus a natural part of the healing process. With the exception of very minor lesions, skin wounds following accidents, disease or surgery all result in some degree of visible scarring. For example, visible scarring may occur as a result of incisions made in breast reduction surgery, C-sections, abdominoplasties, excision of scars, abrasions, accidental cuts and as a result of burns.

In cases where the scar tissue is large or in a prominent location, it can be quite apparent to a casual observer and thus embarrassing or otherwise problematic for the person who is scarred. Variables that may affect the severity of scarring include the depth of the wound at the scar site, the blood supply to the area, the thickness and color of the skin at the site, and the directionality of the scar. A person's age may also affect scar formation in that younger skin tends to produce thicker scars than older skin. Also, scars are more noticeable on tighter, thinner skin areas. The problem may be compounded where scar tissue darkens and becomes thicker and more apparent over time. In addition to the aesthetic and psychological issues, scarring may be problematic where the scar tissue overlays a skeletal joint making movement of the joint painful and restricted. Finally, scar tissue may be accompanied during healing and thereafter by itching or pain related to the thickness of the scars, with thicker scars being associated with increased such distress.

The overgrowth of dense fibrous tissue that typically develops after healing of a skin injury is generally referred to as a keloid scar. The keloid tissue typically extends to and beyond the borders of the original wound and does not regress spontaneously. Hypertrophic scars, on the other hand, typically occur after thermal injuries and other injuries that involve the deep dermis, and are characterized by erythematous, pruritic, raised fibrous lesions that usually do not extend beyond the boundaries of the initial injury and may undergo partial spontaneous resolution over time. Both keloid and hypertrophic scars present the physical and psychological issues outlined above.

Silicone gel sheets have been used in the past in addressing the formation of hypertrophic scars and keloids and for treating existing hypertrophic scars and keloids. Their use, however, has drawbacks. Conventional medical tapes are unusable to maintain the silicone sheets in place because they do not stick to silicone and therefore silicone gel sheets are difficult to keep in place over a wound site or a scar. Additionally, the exposed outer surface of the sheets is tacky to the touch so that articles of clothing tend to adhere to the gel sheets.

More importantly, however, the use of conventional silicone sheets for this purpose may lead to infection as a result of the growth of microbes on the skin, particularly in surgical or post-traumatic wounds in which epidermal integrity has been lost (the skin has broken). As a result, the application of conventional silicone sheets typically must be delayed for several days or longer after the initial wound formation or until after the wound has already begun healing and is less susceptible to infection. However, such delayed use of the silicone sheeting increases the likelihood of the formation of raised keloid or hypertrophic scars seriously compromising the remediation of scarring otherwise achievable with silicone sheets.

Additionally, there is often redness, bruising and swelling associated with the trauma that produces the initial wound. This bruising and swelling can be unsightly and painful. Silicone gel sheets offer no assistance to resolving such bruising and swelling.

The risk of infection or irritation associated with application of the silicone sheets immediately after wound formation can be minimized by physician monitoring in multiple visits, particularly during the first few weeks after wound creation, but this is labor intensive and may be prohibitively expensive. Also, a topical antibiotic medicament (cream, ointment or lotion) could be used to control infection. Although, topical medicaments will control the risk of dermatitis or infection, they have no efficacy in scar improvement. Indeed, common topical ointments and creams, such as topical antibiotics are common causes of allergic contact dermatitis, or allergy. This can result in red, swollen, or even eroded or blistered skin, which may potentially worsen the scar or at least slow wound healing and temporarily worsen scar appearance. Additionally, the application of a topical antibiotic medicament between the silicone sheet and the wound site presents drawbacks because it results in impaired adhesion and lack of firm apposition of the silicone gel sheeting to the wound. The required application of a topical antibiotic medicament is also time-consuming for the patient who has to frequently remove the silicone sheet, reapply the medicament, and reapply the silicone gel sheet.

There is thus a need for a wound treatment that can be applied immediately after the wound is created and that can be maintained in place until the wound is healed without significant danger of infection to produce substantial improvements in the healing of the wound, in the remediation of bruising and swelling at the wound site, and in the visual appearance of the final mature scar. There is also a need for a wound treatment to flatten scars, reduce discomfort as healing proceeds, and remediate scarring without the repeated applications of topical medicaments.

### BRIEF SUMMARY OF THE INVENTION

The present invention includes an occlusive wound dressing for application to wound sites to accelerate the healing process and improve the appearance of the final mature scar. The wound dressing includes a silicone gel sheet with antimicrobial silver disposed in the silicone gel sheet.

The antimicrobial silver may be concentrated near a surface of the silicone gel sheet to be applied to the wound site. Also, the antimicrobial silver may be disposed in the silicone gel sheet in a grid pattern. Alternatively, the antimicrobial silver may be present in the form of a lattice of silver wires.

Adhesive may be present on at least one surface of the silicone gel sheet to be applied to the wound site. This adhesive may be applied uniformly across the surface of the gel sheet or it may be in the form of spaced lines with adhesive-free areas between the lines containing the antimicrobial silver. Alternatively, where the antimicrobial silver is present in the form of a lattice of silver wires the adhesive may be positioned within the surface areas between the wires.

In an important embodiment of the present invention, an antibruising agent is disposed in the silicone gel sheet. The antibruising agent may be Arnica, vitamin K, or another appropriate antibruising agent.

In an alternative embodiment, a semi-solid silicone gel containing antimicrobial silver dispersed therein may be used in lieu of the silicone gel sheet. After the semi-solid silicone gel is applied to the wound site, an adhesive tape is applied over the gel to maintain it in place.

In yet another alternative embodiment of the invention, the silicone gel sheet is replaced by a semi-solid moldable silicone gel which includes a heat or light-sensitive hardening agent. This moldable silicone gel is applied and molded over the wound site and then subjected to heat or light to harden the gel and fix it in place. The intrinsic adhesive function of the moldable silicone may hold it in place depending on the size and shape of the wound area. If desired, tape could be applied to the molded gel to hold it in place or adhesive could be applied to the skin before applying the moldable gel.

Further advantages and characteristics of the present invention will become apparent to those skilled in the art from the detailed description that follows and the accompanying drawings. Preferred but non-exclusive embodiments of the invention are illustrated and discussed by way of non-limiting examples of the invention.

### BRIEF DESCRIPTIONS OF THE DRAWINGS

Preferred embodiments of the invention are described with reference to the accompanying drawings, in which like elements bear like reference numerals, and wherein:

FIG. 1 is a perspective view of a silicone sheet in accordance with the present invention;

FIG. 2 is a top plan view of a silicone sheet in accordance with the present invention in which antimicrobial silver particles are disposed in a grid pattern;

FIG. 3 is a top plan view of a silicone sheet in accordance with the present invention in which silver wires are disposed in a lattice in the sheet;

FIG. 4 is a perspective view of a silicone sheet in accordance with the present invention in which adhesive is applied to the surface of the sheet in thin spaced lines with adhesive-free areas between the lines containing accessible antimicrobial silver; and

FIG. 5 is a silicone sheet in accordance with the present invention where adhesive is applied across the sheet except for a central adhesive-free area.

### DETAILED DESCRIPTION OF THE INVENTION

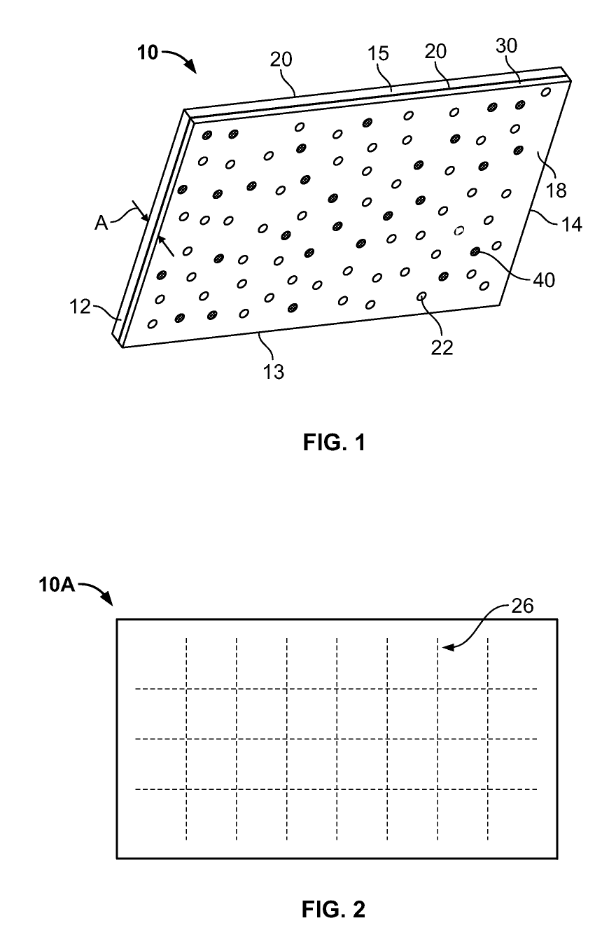

Turning to FIG. 1, a silicone gel sheet 10 is shown in cross-section with a thickness “A,” which is exaggerated for purposes of illustration. The silicone sheet has edges 12, 13, 14 and 15, a bottom side 16, and a top side 18 (to be applied to the wound). Silicone sheet 10 comprises silicone gel 20. The term “silicone gel” as used herein may mean a cured silicone gel composed mainly of organopolysiloxane having a low crosslinking density and an appropriate penetration and hardness. The silicone gel sheet may have a surface that accepts colorization or it may be made available in various skin tone colors corresponding to common skin shades.

In a preferred embodiment, the silicone sheet will be about 0.5-2 mm in thickness. At this thickness the sheet will be lightweight and easily bent to facilitate smooth apposition to irregular skin surfaces (e.g., the face surface) and it will be unlikely to be visible when the area of the wound site is viewed by bystanders. Also, the silicone sheet may be provided in varying sizes for application to varying wound sizes or in larger sheets capable of being trimmed to the desired size and shape using a scissors or other cutting implement. For example, non-limiting preferred sheet sizes include strips about 1-2 inches long and about 0.5-1 inch wide, or rectangular or square sheets with side lengths of 3-5 inches, or oval sheets generally of like dimensions.

Silicone gel 20 is embedded with elemental silver or silver salts (referred to below as “antimicrobial silver”) which will release silver ions when in contact with the wound site. The antimicrobial silver is represented diagrammatically by particles 22. The positively charged ionic form of silver has antimicrobial activity against a broad spectrum of relevant microorganisms and is effective in small quantities. Thus, any effective form of antimicrobial silver (including colloidal and nanoparticle forms) may be used and should be present at a level that will produce the desired antimicrobial effect.

The presence of antimicrobial silver enables the silicone gel sheet to be applied to early healing wounds while minimizing the risk of inducing infection. Indeed, the antimicrobial effectiveness of the antimicrobial silver is enhanced by the direct contact of the surface of the silicone sheet with the wound site. This also minimizes or eliminates opportunities for growth in non-contact open areas of the wound. The antimicrobial silver preferably will be concentrated near the surface of the silicone gel sheet on the side of the silicone gel sheet to be applied to the wound, e.g., top side 18 in FIG. 1. When the antimicrobial silver is provided in the form of particles of elemental silver, the silver particles preferably will not protrude from surface 18 of the sheet since protruding particles risk a friction injury to the wound site and may interfere with the firm apposition of the silicone sheet to the wound.

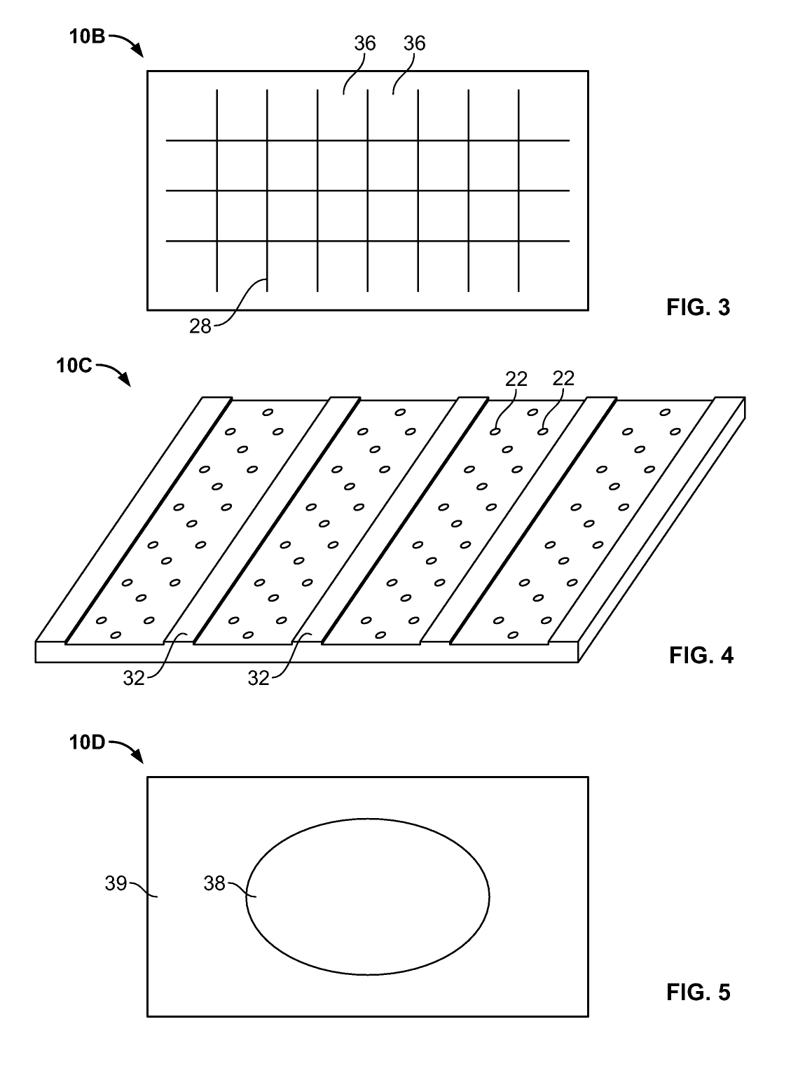

In one embodiment of the invention, the antimicrobial silver particles may be disposed through the silicone gel in a uniform manner. Alternatively, they may be disposed in silicone sheet 10A in a grid pattern 26 as shown in FIG. 2. In yet another alternative, the elemental silver may be present in silicone sheet 10B in the form of fine silver wires 28 disposed, e.g., in the form of a wire lattice as shown in FIG. 3 or the antimicrobial silver may be disposed as otherwise desired. In the wire lattice pattern of FIG. 3, it is preferred that the silver wires have a diameter of from about 0.01 to 1.0 mm and that they be spaced 0.1 to 2.0 mm apart. Also, preferably the wires will be disposed flush to the surface of the silicone sheet to ensure close contact with the wound and optimal antibiotic effect. If the wires are spaced from the wound, it is preferred that there be substantially nothing except air/fluid (i.e., no silicone) between the wires and the skin surface.

As noted above, in one embodiment of the invention, the antimicrobial silver may be distributed in a uniform manner throughout the silicone gel sheet. This results in a double-sided silicone sheet that may be applied with appropriate adhesive on one side of the silicone gel sheet which is initially applied to the wound and when the adhesive action (as described below) on that side is exhausted, the silicone gel sheet may be turned over exposing adhesive and antimicrobial silver on the opposite side so that the opposite side of the sheet can be applied to the wound.

Preferably a layer of a long-lasting adhesive 30 will be applied to the silicone sheet on the side(s) intended to be applied to the healing wound (e.g., side 18 in FIG. 1). The adhesive of the silicone sheets splints the wound to reduce tension across it, maximizing the wound healing result and minimize scarring. In order to minimize obstruction of the contact plane between the wound and the silicone gel, the adhesive may be applied as in silicone sheet 10C in thin spaced lines 32 shown in exaggerated thickness in FIG. 4 with the adhesive-free areas 34 between the lines containing accessible antimicrobial silver. In another alternative embodiment, where the silver will be present as lattice of silver wires (FIG. 3), the adhesive may be positioned within the areas 36 between the wires to maximize the amount of accessible elemental silver located outside of the adhesive areas. Finally, the adhesive may be applied as in FIG. 5 where the silicone sheet has a central adhesive-free area 38 for application over the wound site and adhesive 39 located thereabout.

In yet another alternative embodiment of the invention, the adhesive need not be pre-applied. Instead, it may be supplied exogenously and applied either to the surface of the wound area or to the silicone sheet just before application of the sheet to the wound site. Also, silver particles may be included in such an exogenous adhesive and applied to the silicone gel sheet or to the wound site prior to the application of the silicone sheet to the wound site.

Finally, an antibruising agent such as Arnica preferably will be incorporated in the silicone gel to reduce bruising. Arnica, which is also known as Arnica Montana or Leopard's Bane, is an herb that is indigenous to Central Europe. Its constituents include a bitter, yellow crystalline principle, Arnicin, a volatile oil, tannin and phulin, a tincture. The presence of Arnica is illustrated diagrammatically in FIG. 1 by particles 40 although the Arnica will be provided in a molecular form, likely in an herbal extract which may be in an aqueous, gel or other form. For example, the Arnica Montana extract can be used in the form of a tincture or a glycol extract.

The concentration of the Arnica Montana in the silicone sheet will be at a level sufficient to reduce bruising and inflammation and below that which would cause irritation or other adverse effects. Currently, it is believed that the level of active Arnica should be at least about 0.001 percent by weight based on the weight of the silicone gel and preferably will be about 5 to 25 percent by weight based on the weight of the silicone gel. The Arnica may also be applied topically as an ointment in an appropriate vehicle where there is at least a 10% and preferably at least a 20% concentration of Arnica. The Arnica should be available at the wound or scar surface and so should be flush with the silicone surface or at least adjacent the surface, with little or preferably no intervening or impeding barrier. Also, the Arnica may be in the form of pockets or dots on the surface of the gel sheet in order to avoid undermining the therapeutic effect of the silicone gel sheet itself, which should be contiguous with as much of the scar as possible. Where a lattice of silver wires is used to provide antimicrobial action, the Arnica may be placed within the space between the wires. Since bruising typically occurs around the scar, in yet another alternative embodiment the silver wire lattice maybe confined to the center of the silicone sheet (e.g., in area 38 of FIG. 5) for positioning against the wound and the Arnica may be disposed in the area surrounding the lattice. Also, Vitamin K can be used along with the Arnica to help reduce bruising.

When the scars being treated are on the limbs, including legs and arms, preferably, the silicone gel sheet will be held in place with a compressive wrap such as a neoprene wrap or an Ace wrap. The wrap should be easy to apply and remove and may be closeable with a fastener and then tightenable (like a seat belt on a plane). It may also be waterproof so that it doesn't need to be removed prior to bathing and it may be reusable to reduce costs.

In an alternative embodiment of the invention, a semisolid silicone gel can be used instead of a sheet of silicone with silver particles dispersed in the semisolid silicone gel. This gel would be applied to healing wounds without adhesive but would be used in combination with an appropriate tape or band applied over the gel to prevent loss or migration of the gel. Alternatively the silicone gel may be in the form of a semi-solid moldable form applied and molded to the wound site where a heat or light sensitive hardening agent is present in the gel. In this case, the moldable gel would be applied to the wound area and then subjected to heat or light to fix and harden its shape.

Finally, since it is desirable to start using wound dressings soon after injury or surgery when such sheets are most likely to prevent keloid or hypertrophic scar formation, it is also desirable to incorporate an antibiotic in the scar dressing to prevent infection.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

What is claimed is:

1. An occlusive wound dressing for application to wound sites to accelerate the healing process and improve the appearance of the final mature scar comprising:
a silicone gel sheet; and
antimicrobial silver disposed in the silicone gel sheet.

2. The occlusive wound dressing of claim 1 in which the antimicrobial silver is concentrated near a surface of the silicone gel sheet to be applied to the wound sites.

3. The occlusive wound dressing of claim 1 in which antimicrobial silver is disposed in the silicone gel sheet in a grid pattern.

4. The occlusive wound dressing of claim 1 in which antimicrobial silver is present in the form of silver wires disposed in a lattice pattern.

5. The occlusive wound dressing of claim 4 in which the silver wires are disposed in a lattice pattern.

6. The occlusive wound dressing of claim 4 in which the silver wires have a diameter of from about 0.1 to 1.0 mm.

7. The occlusive wound dressing of claim 5 in which the wires are spaced about 0.1 to 2.0 mm apart.

8. The occlusive wound dressing of claim 1 in which an adhesive layer is present on at least one surface of the silicone gel sheet to be applied to the wound sites.

9. The occlusive wound dressing of claim 8 in which the adhesive layer is present in the form of spaced lines with adhesive-free areas between the lines containing the antimicrobial silver.

10. The occlusive wound dressing of claim 8 in which antimicrobial silver is present in the form of silver wires and the adhesive is positioned within the areas between the wires.

11. The occlusive wound dressing of claim 1 including a separately supplied adhesive for application either to the surface of the wound area or to the silicone gel sheet just before application of the sheet to the wound site.

12. The occlusive wound dressing of claim 11 in which antimicrobial silver particles are included in the adhesive.

13. The occlusive wound dressing of claim 1 including an antibruising agent disposed in the silicone gel sheet.

14. The occlusive wound dressing of claim 13 in which the antibruising agent is Arnica.

15. The occlusive wound dressing of claim 14 in which the level of Arnica is at least about 0.001 percent by weight based on the weight of the silicone gel.

16. The occlusive wound dressing of claim 14 in which the antibruising agent vitamin K is also present.

17. The occlusive wound dressing of claim 1 including a compressive wrap for holding the silicone gel sheet in place at the wound site.

18. The occlusive wound dressing of claim 1 including a separately supplied Arnica ointment containing at least 10% by weight Arnica for application at the wound site before silicone gel sheet.

19. An occlusive wound dressing for application to a wound site to accelerate the healing process and improve the appearance of the final mature scar comprising:
a semi-solid silicone gel having antimicrobial silver particles dispersed therein for application to the wound site; and
an adhesive tape for application over the gel.

20. An occlusive wound dressing for application to a wound site to accelerate the healing process and improve the appearance of the final mature scar comprising:
a semi-solid moldable silicone gel including antimicrobial silver particles dispersed therein and a heat or light sensitive hardening agent for application and molding over the wound site before being subjected to heat or light to fix and harden its shape.
